(19) United States
(12) Patent Application Publication
(10) Pub. No.: US 2015/0366718 A1
(43) Pub. Date: Dec. 24, 2015

(54) THREE DIMENSIONAL WOUND DRESSING

(71) Applicant: BIO-MEDICAL CARBON TECHNOLOGY CO., LTD., Taichung City (TW)

(72) Inventors: Tse-Hao KO, Taipei City (TW); Jui-Hsiang LIN, Taichung City (TW); Pei-Hsun CHOU, Taichung City (TW); Yen-Ju SU, Taichung City (TW); Wei-Shan HSU, Taichung City (TW)

(21) Appl. No.: 14/313,536

(22) Filed: Jun. 24, 2014

(51) Int. Cl. A61F 13/00

(52) U.S. Cl. CPC A61F 13/00068

(57) ABSTRACT

A three dimensional wound dressing includes a foam body having a planar bottom surface and a convex arc-shaped top surface. The three dimensional wound dressing can be placed into a cavity wound in such a way that the top surface is abutted against an inner wall and a bottom of the cavity wound to effectively absorb wound exudate therefrom. Because the integrity of the foam body of the wound dressing can be maintained after the wound dressing absorbs the wound exudate, a user or a medical staff can conveniently remove and replace the wound dressing.

### BACKGROUND OF THE INVENTION

1. Field of the Invention

The present invention relates generally to a wound dressing and more particularly, to a three dimensional (3D) wound dressing capable of absorbing wound exudate from a cavity wound and can be conveniently replaced by a user.

2. Description of the Related Art

A cavity wound means a concave wound or a chamber wound caused by tissue loss. Clinically, the cavity wound can be divided into four types: a surgical wound from local tissue excision, a surgical wound from a wound infected by bacteria and then suffered from pus discharge, an acute wound requiring debridement, and a chronic wound such as stage three or four pressure sore, diabetic skin ulcer, and diabetic foot ulcer.

An ideal wound dressing for nursing a cavity wound should have the following properties: (i) can be placed in the chamber and can be in contact with the bottom of the wound, (ii) can absorb large amount of exudate, (iii) maintains its integrity after absorbing exudate for convenient remove, and (iv) is easy and comfortable to remove/change. Conventionally, the material used to nurse cavity wound is calcium alginate which is soft and easy to be placed in the cavity wound, has good absorbing capacity, and can be in contact with the bottom of the cavity wound to absorb wound exudate. However, because the calcium alginate wound dressing transforms into gel form after absorbing wound exudate, it cannot be removed integrally by a medical staff. As a result, the wound should be cleaned several times to remove the gel left over the wound thereby wasting time and increasing the cost of medical staff.

In addition, the Aquacel® wound dressing is also available on the market, which has a superior water-absorbing capacity and has been widely used for cavity wound clinically. However, because the Aquacel® wound dressing also transforms into gel form after absorbing wound exudate, the wound should be cleaned several times to remove the gel left over the wound upon replacing a new wound dressing, thereby increasing the burden of medical staff.

Foam material applied for wound dressing is also a material with high absorbing capacity. However, because the commercially available foam wound dressing is a planar sheet, it is not easy to be filled in the cavity wound and cannot be fitted tightly to the inner wall of the cavity wound although the foam wound dressing has high absorbing capacity and can maintain its integrity after absorbing the wound exudate.

### SUMMARY OF THE INVENTION

The main objective of the present invention is to provide a 3D wound dressing that can effectively absorb wound exudate from a cavity wound and can be conveniently replaced by a user.

In order to achieve the aforesaid objective, the 3D wound dressing of the present invention includes a foam body having a planar bottom surface and a convex arc-shaped top surface. The 3D wound dressing of the present invention can be placed into a cavity wound in such a way that the top surface is abutted against an inner wall and a bottom of the cavity wound to effectively absorb wound exudate therefrom. The integrity of the foam body of the wound dressing can be maintained after the wound dressing absorbs the wound exudate, thereby assisting a user or a medical staff to conveniently remove and replace the wound dressing.

### BRIEF DESCRIPTION OF THE DRAWINGS

The present invention will become more fully understood from the detailed description given herein below and the accompanying drawings which are given by way of illustration only, and are not limitative of the present invention, and wherein:

FIG. 1 is a top view of a 3D wound dressing according to a first preferred embodiment of the present invention;

FIG. 2 is a lateral side view of the 3D wound dressing of the first preferred embodiment of the present invention;

FIG. 3 is a schematic diagram showing that the 3D wound dressing of the first preferred embodiment is placed into a wound;

FIG. 4 is a top view of a 3D wound dressing according to a second preferred embodiment of the present invention;

FIG. 5 is a front view of the 3D wound dressing according to the second preferred embodiment of the present invention; and

FIG. 6 is a right side view of the 3D wound dressing according to the second preferred embodiment of the present invention.

### DETAILED DESCRIPTION OF THE INVENTION

It will be appreciated that in the preferred embodiments to be detailed described hereunder, the same reference numeral represents the same or similar part.

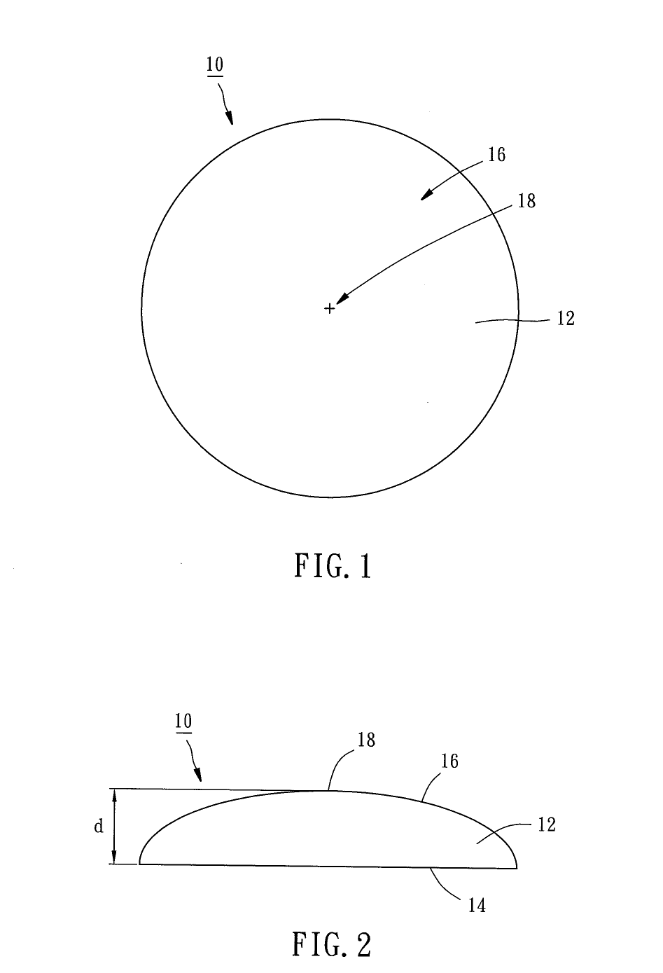

FIGS. 1 and 2 show the 3D wound dressing 10 provided by a first preferred embodiment of the present invention. The 3D wound dressing 10 includes a foam body 12. The foam body 12 is foamed from, but not limited to, polyurethane (PU) in the first embodiment. In fact, the foam body 12 may be made from a material selected from polyethylene (PE), polyvinyl alcohol (PVA) or ethylene vinyl acetate (EVA) in the actual manufacture of the foam body 12.

The foam body 12 has a planar bottom surface 14 and an arc-shaped top surface 16 protruding upward, such that the foam body 12 is shaped like a hemispheroid. The bottom surface 14 is substantially circle-shaped and has a diameter of about 10 cm. The top surface 16 has an apex 18 and a first distance d between the apex 18 and the bottom surface 14 is larger than a second distance between a location of the top surface 16 other than the apex 18 and the bottom surface 14. The first distance d is preferably 2 cm.

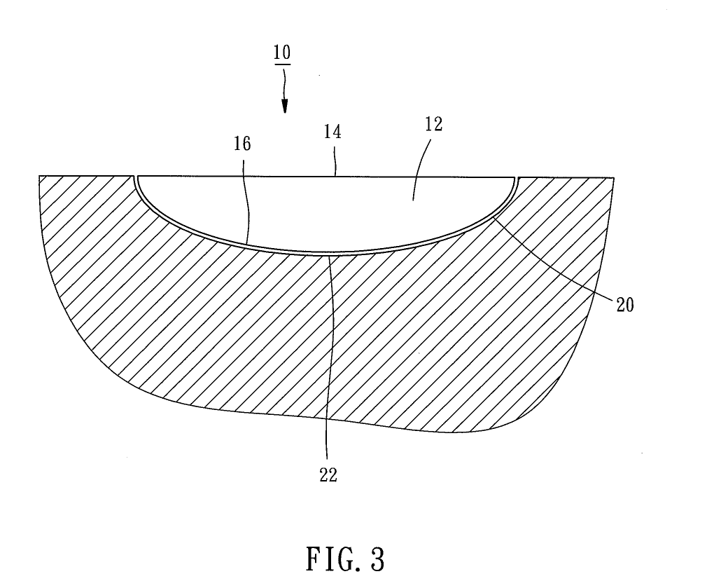

In this embodiment, the foam body 12, which is foamed from a material with high liquid-absorbing property, is configured having a hemispheroid-shaped. As shown in FIG. 3, when the foam body 12 is placed into a cavity wound 20 in a way that the top surface 16 faces the cavity wound 20, the 3D wound dressing 10 can be in contact with a bottom 22 and an inner wall of the wound 20 to absorb the wound exudate. In the first preferred embodiment, the foam body 12 made from PU has a liquid-absorbing capacity of 2.9 mL/cm³ to 3.2 mL/cm³.

Because the integrity of the foam body 12 can be maintained after the foam body 12 absorbs wound exudate, in comparison with the existing wound dressing, the 3D wound dressing 10 can be easily removed and the times for cleaning the wound can be reduced during the replacement process. Therefore, the 3D wound dressing 10 of the present invention can more effectively absorb wound exudate to reduce the medical care cost, and is especially suitable for the care of a large-area cavity wound such as pressure sore, diabetic ulcer and the wound after debridement, and chronic cavity wound.

According to the design concept of the present invention, the 3D wound dressing 10 may be configured having a variety of sizes for different-sized wounds. For example, the bottom surface 14 may have a diameter ranging from 2 cm to 30 cm, and the distance d from the apex 18 to the bottom surface 14 may range from 1 cm to 10 cm.

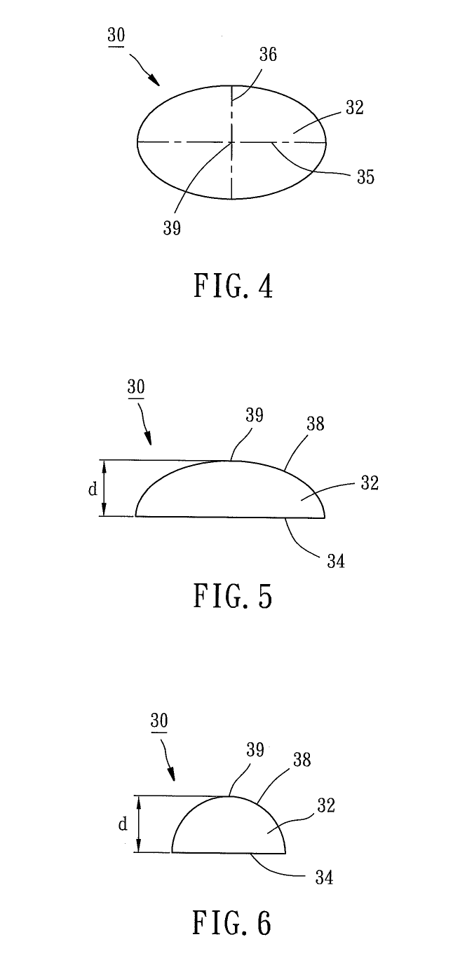

In addition, the 3D wound dressing 10 may be designed having a variety of shapes. As shown in FIGS. 4 to 6, the 3D wound dressing 30 provided by a second preferred embodiment of the present invention has a semiellipsoid-shaped foam body 32. The bottom surface 34 is substantially oval-shaped and has a major axis 35 and a minor axis 36. The ratio of the length of the major axis 35 to the length of the minor axis 36 of the bottom surface 34 is preferably 5:3. For example, the major axis 35 has a length of 5 cm and the minor axis has a length of 3 cm. The top surface 38 has an apex 39 and a distance d between the apex 39 and the bottom surface 34 is preferably 1.5 cm.

The 3D wound dressing 30 of the second preferred embodiment is suitable for an elongated cavity wound such as the wound after local tissue resection or the surgery wound after the stitches are removed.

The 3D wound dressing 30 may be provided having various sizes subject to different-sized wounds. For example, the length ratio of the major axis 35 to the minor axis 36 of the bottom surface 34 may be in a range of 5:4 to 5:1, the major axis 35 may have a length ranging from 2 cm to 30 cm, and the distance d from the apex 39 to the bottom surface 34 may be in a range of 1 cm to 10 cm.

It is worth mentioning that the wound dressing of the present invention can be trimmed to match the actual shape of the wound, and then the trimmed wound dressing can be compressed into the wound by utilizing the elastic property of the foam body, such that the 3D wound dressing of the present invention has a relatively higher practicability in comparison with the existing planar wound dressing. Further, because the wound dressing of the present invention can be fitted tightly to the inner wall of the wound, an excellent wound exudate absorbing effect can be achieved. Furthermore, since the bottom surface of the wound dressing of the present invention is a planar surface, it may be flush with the periphery of the skin when the wound dressing is placed in the wound. As a result, the wound dressing can be conveniently bandaged or fixed by an adhesive type and can prevent unexpected detachment of the wound dressing. Therefore, although the configuration of the 3D wound dressing of the present invention is not complicated, the 3D wound dressing of the present invention is a practical and excellent design actually.

1. A three dimensional wound dressing for being placed in a cavity wound, the three dimensional wound dressing, comprising:
a foam body having a planar bottom surface and a convex arc-shaped top surface;
wherein the three dimensional wound dressing is adapted for being placed in the cavity wound so that the convex arc-shaped top surface faces the cavity would.

2. The three dimensional wound dressing as claimed in claim 1, wherein said bottom surface has a shape of circle.

3. The three dimensional wound dressing as claimed in claim 2, wherein said bottom surface has a diameter ranging from 2 cm to 30 cm; said top surface has an apex; a first distance between said apex and said bottom surface, which ranges from 1 cm to 10 cm, is larger than a second distance between said bottom surface and a location of said top surface other than said apex.

4. The three dimensional wound dressing as claimed in claim 1, wherein said bottom surface has a shape of oval.

5. The three dimensional wound dressing as claimed in claim 4, wherein said bottom surface has a major axis and a minor axis and a ratio of a length of said major axis to a length of said minor axis ranges from 5:4 to 5:1; the length of said major axis ranges from 2 cm to 30 cm; said top surface has an apex; a first distance between said apex and said bottom surface, which ranges from 1 cm to 10 cm, is larger than a second distance between said bottom surface and a location of said top surface other than said apex.

6. The three dimensional wound dressing as claimed in claim 1, wherein said foam body is made from a material selected from the group consisting of polyurethane, polyethylene, polyvinyl alcohol, and ethylene vinyl acetate.

7. The three dimensional wound dressing as claimed in claim 1, wherein the three dimensional wound dressing is in contact with a bottom and an inner wall of the cavity wound.
